(19) United States
(12) Patent Application Publication
(10) Pub. No.: US 2022/0387594 A1
(43) Pub. Date: Dec. 8, 2022

(54) USE OF AMITRIPTYLINE AND/OR ONE OF THE PHARMACEUTICALLY ACCEPTABLE SALTS THEREOF AS A PRESERVATIVE

(71) Applicant: AlgoTherapeutix, Suresnes (FR)

(72) Inventors: Frédéric LALLEMAND, Rambouillet (FR); Philippe PICAUT, Verrieres Le Buisson (FR)

(73) Assignee: AlgoTherapeutix, Suresnes (FR)

(21) Appl. No.: 17/831,276

(22) Filed: Jun. 2, 2022

(30) Foreign Application Priority Data
Jun. 3, 2021 (FR) 2105871

(51) Int. Cl. A61K 47/18; A61K 47/10

(52) U.S. Cl. CPC A61K 47/18; A61K 47/10

(57) ABSTRACT

The present invention relates to a method for the preservation of a pharmaceutical composition by adding amitriptyline and/or one of the pharmaceutically acceptable salts thereof.

### CROSS-REFERENCE TO RELATED APPLICATION

This application claims priority to French Application No. 2105871, filed Jun. 3, 2021, the entire disclosure of which is incorporated by reference herein.

### BACKGROUND

The present invention relates to the use of amitriptyline and/or one of the pharmaceutically acceptable salts as a preservative in a pharmaceutical composition.

The pharmaceutical compositions and the substances they contain are subject to significant standards, particularly in terms of the presence of microorganisms, such as bacteria, yeasts or fungi, in order to avoid the risk of infection for the user and the deterioration of the pharmaceutical compositions.

In Europe, Chapter 5.1.4 “Microbiological quality of the pharmaceutical preparations” of the European Pharmacopoeia indicates that the topically administered formulations do not need to be sterile. However, they must not comprise more than one hundred microorganisms per gram of composition and they must not comprise Staphylococcus aureus, nor Pseudomonas aeruginosa.

In order to verify their proper compliance with these standards before they are placed on the market, the compositions should be subjected to tests such as those specified by monographs 2.6.12 and 2.6.13 requiring an enumeration of germs and microorganisms and setting a limit threshold not to exceed.

In order to meet these standards and maintain such a low microorganism population, it is often necessary to incorporate preservatives or antimicrobial agents into the pharmaceutical compositions. Chapter 5.1.3 “Effectiveness of antimicrobial preservation” of the European Pharmacopoeia clearly defines how the effectiveness of an antimicrobial preservation in a pharmaceutical product must be assessed.

Such standards and such tests are also in force in the United States (USP specifications, Chapter 51).

Among the preservatives commonly used in the pharmaceutical compositions, there are in particular parabens, such as methyl paraben. However, these compounds are suspected of interacting with the receptors of certain hormones and therefore of having a role as endocrine disruptors.

There is therefore a real need to provide new antimicrobial preservatives which can be used in the pharmaceutical compositions, in particular the topical pharmaceutical compositions.

It has been surprisingly discovered that amitriptyline and/or one of the pharmaceutically acceptable salts thereof would allow obtaining an antimicrobial effect equivalent to a paraben-type preservative in pharmaceutical compositions.

The present invention relates to a method for the preservation of a pharmaceutical composition, preferably for inhibiting the growth of microorganisms such as bacteria, yeasts and/or fungi in a pharmaceutical composition, comprising the addition, into said pharmaceutical composition, of amitriptyline and/or one of the pharmaceutically acceptable salts thereof.

The use of amitriptyline and/or one of the salts thereof allows formulating pharmaceutical compositions with a small, or even zero, amount of paraben-type preservative. Such a use therefore allows limiting the presence of potential endocrine disruptors or preservatives having risks of causing allergies or irritations.

The present invention also relates to a pharmaceutical composition as defined below comprising amitriptyline and/or one of the pharmaceutically acceptable salts thereof, preferably in a total content comprised between 1 and 25% by weight, more preferably between 5 and 20% by weight, more preferably still between 5 and 15% by weight, most preferably between 10 and 15% by weight, relative to the total weight of the pharmaceutical composition.

Other objects, features, aspects and advantages of the invention will appear even more clearly on reading the description and the following example.

In the present description, and unless otherwise indicated:
the expression “at least one” is equivalent to the expression “one or more” and may be substituted therefor;
the expression “comprised between . . . and . . . ” is equivalent to the expression “ranging from . . . to . . . ” and can be substituted therefor, and implies that the limits are included;
the expression “polyoxyalkylene” corresponds, within the meaning of the invention, to an —(O-alkyl)n- unit, where n is an integer ranging from 2 to 200, preferably from 2 to 40, more preferably from 2 to 20;
the expression “polyoxyethylenated” corresponds, within the meaning of the invention, to a pattern —(O—CH₂CH₂)ₙ—, where n is an integer ranging from 2 to 200, preferably from 2 to 40, more preferably from 2 to 20;
the expression “the composition is free of a compound” means that the composition contains less than 2% by weight of said compound, preferably less than 1% by weight of said compound, more preferably less than 0.5% by weight of said compound, better less than 0.1% by weight, better still less than 0.05% by weight, relative to total weight of the composition, and even better a composition which does not contain said compound. In this type of composition, any “compound” present is not added during the preparation of the composition but corresponding to the residual “compound” provided by the mixed ingredients.

Amitriptyline and the Salts Thereof

The method according to the present invention implements at least amitriptyline and/or one of its pharmaceutically acceptable salts in a pharmaceutical composition.

Amitriptyline has the following formula (I):

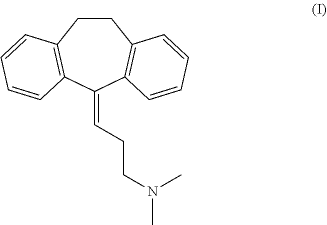 [SMILES: CN(C)CCC=C1c2ccccc2CCc2ccccc21]

Within the scope of the present invention, the term “pharmaceutically acceptable salts of amitriptyline”, means the salts compatible with a pharmaceutical composition, that is to say intended to be administered to humans. In particular, the term “pharmaceutically acceptable salt of amitriptyline” means the hydrates, the solvates, the salts of acids such as the hydrochlorides and the clathrates of amitriptyline.

As a particularly preferred salt of amitriptyline, amitriptyline hydrochloride is used.

Preferably, the total content of amitriptyline and/or one of the pharmaceutically acceptable salts is comprised between 1 and 25% by weight, preferably between 5 and 20% by weight, more preferably still between 5 and 15% by weight, most preferably between 10 and 15% by weight, relative to the total weight of the pharmaceutical composition.

More preferably, the total content of amitriptyline hydrochloride is comprised between 1 and 25% by weight, more preferably still between 5 and 20% by weight, even better between 5 and 15% by weight, most preferably between 10 and 15% by weight, relative to the total weight of the pharmaceutical composition.

The presence of amitriptyline and/or one of the pharmaceutically acceptable salts thereof in the pharmaceutical composition according to the invention allows obtaining a composition which complies with the criteria of the European and American Pharmacopoeia, in particular relative to the presence of microorganisms in the composition. In particular, the presence of amitriptyline and/or one of the pharmaceutically acceptable salts thereof allows limiting, or even preventing, the growth of microorganisms such as Pseudomonas aeruginosa, Staphylococcus aureus, Escherichia coli, Candida albicans, Aspergillus brasiliensis.

The composition may optionally further comprise at least one additional preservative, different from amitriptyline and the salts thereof, such as parabens (e.g., methyl paraben). In this case, the content of additional preservative(s) is preferably less than or equal to 10% by weight, more preferably less than or equal to 5% by weight, more preferably still less than or equal to 1% by weight, most preferably less than or equal to 0.5% by weight, relative to the total weight of the pharmaceutical composition.

The composition according to the invention may optionally further comprise one or more additional preservatives, which are different from amitriptyline and the salts thereof, chosen from methyl paraben, ethyl paraben, propyl paraben, butyl paraben, benzalkonium chloride, potassium sorbate, phenoxyethanol, chlorhexidine, benzyl alcohol, boric acid, butylene glycol, cetrimide, chlorobutanol, sodium benzoate, and mixtures thereof.

In a preferred embodiment, the composition according to the invention is free of additional preservative. In other words, amitriptyline and/or one of the pharmaceutically acceptable salts thereof is(are) the only preservative(s) in the composition according to the invention. In particular, preferably, the composition according to the invention is free of paraben, such as methyl paraben, ethyl paraben, propyl paraben, butyl paraben, benzalkonium chloride, potassium sorbate, phenoxyethanol, chlorhexidine, benzyl alcohol, boric acid, butylene glycol, cetrimide, chlorobutanol, sodium benzoate.

Water

The composition according to the present invention may comprise water.

Preferably, the total water content is greater than or equal to 75% by weight, more preferably between 75 and 95% by weight; more preferably still between 75 and 90% by weight, relative to the total weight of the composition.

Polyols

Preferably, the composition according to the present invention may further comprise at least one C₂-C₈ polyol.

The term “C₂-C₈ polyol” within the meaning of the present invention, is means an organic compound consisting of a C₂-C₈ hydrocarbon chain, optionally interrupted by one or more oxygen atoms, and carrying at least two free hydroxyl groups (—OH) carried by different carbon atoms, this compound possibly being cyclic or acyclic, linear or branched, saturated or unsaturated, and in the liquid state at ambient temperature (25° C.) and at atmospheric pressure (i.e. 1.013.10⁵ Pa).

Preferably, the C₂-C₈ polyol(s) according to the invention are acyclic and non-aromatic.

The C₂-C₈ polyols according to the invention comprise, in the structure thereof, 2 to 8 carbon atoms, preferably 2 to 6 carbon atoms, more preferably 2 to 5 carbon atoms.

More particularly, the polyol(s) that can be used according to the invention comprise 2 to 10 hydroxy groups, more preferably 2 to 5 hydroxy groups, more preferably still 2 to 3 hydroxy groups.

Preferably, said C₂-C₈ polyol(s) which can be used according to the invention are selected from C₃-C₆ polyols, ethylene glycol, and mixtures thereof.

Preferably, the polyols are different from the additional preservative(s) optionally present in the composition.

According to a preferred embodiment of the invention, said C₂-C₈ polyol(s) which can be used according to the invention are selected from propylene glycol, 1,3-propanediol, 1,3-butylene glycol, pentane-1,2-diol, dipropylene glycol, hexylene glycol, pentylene glycol, glycerol, ethylene glycol, and a mixture of these compounds; more preferably the composition comprises at least propylene glycol.

Preferably, when the composition according to the invention comprises at least one C₂-C₈ polyol, the total content of C₂-C₈ polyol(s) is comprised between 0.1 and 15% by weight, more preferably between 0.5 and 10% by weight, more preferably still between 1 and 6% by weight, and even most preferably between 3 and 6% by weight, relative to the total weight of the composition.

Preferably, when the composition according to the invention comprises at least one C₂-C₈ polyol, the total propylene glycol content is comprised between 0.1 and 15% by weight, more preferably between 0.5 and 10% by weight, more preferably still between 1 and 6% by weight, and even most preferably between 3 and 6% by weight, relative to the total weight of the composition.

The Thickening Agents

The composition according to the invention may further comprise at least one thickening agent.

More preferably, the thickening agent(s) are thickening polymers.

The term “thickening polymer” means, according to the present invention, polymers which increase, by their presence at a concentration of 0.05% by weight, the viscosity of the cosmetic compositions into which they are introduced by at least 20 cps (20 mPa·s), preferably by at least 50 cps (50 mPa·s), at ambient temperature (25° C.), at atmospheric pressure and a shear rate of is-1 (the viscosity can be measured using a cone/plate viscometer, Haake R600 rheometer or similar).

By way of example, mention may be made as thickening agent of: homopolymers or copolymers of crosslinked acrylic or methacrylic acid, crosslinked homopolymers of 2-acrylamido-2-methyl-propanesulfonic acid and the crosslinked acrylamide copolymers thereof, ammonium acrylate homopolymers or ammonium acrylate and acrylamide copolymers, cellulosic polymers, and mixtures thereof.

Among the crosslinked acrylic acid homopolymers, mention may be made of those crosslinked with an allyl alcohol ether of the sugar series, such as for example the products sold under the names CARBOPOLS 980, 981, 954, 2984 and 5984 by the company NOVEON or the products sold under the names SYNTHALEN M and SYNTHALEN K by the company 3 VSA. These polymers have the name INCI Carbomer. The thickening polymers can also be crosslinked (meth)acrylic acid copolymers such as the polymer sold under the name AQUA SF1 by the company NOVEON.

More preferably still, the composition according to the invention further comprises at least one cellulosic polymer.

The term “cellulosic” polymer means according to the invention any polysaccharide compound, substituted or not, having in the structure thereof sequences of glucose residues linked via β-1,4 bonds; besides the unsubstituted celluloses, the cellulose derivatives can be anionic, cationic, amphoteric or non-ionic.

Thus, the cellulosic polymers which can be used according to the invention can be selected from the unsubstituted celluloses, including in a microcrystalline form, and the substituted celluloses.

More preferably, the cellulosic polymers that can be used according to the invention do not contain a C₁₀-C₃₀ side fatty chain in the structure thereof.

Preferably, the cellulosic polymer(s) which can be used according to the invention have an average molecular weight comprised between 5,000 and 1,500,000, more preferably between 50,000 and 800,000, more preferably still between 400,000 and 800,000.

Among the cellulosic polymers according to the invention, cellulose ethers, cellulose esters and cellulose ether esters can be distinguished.

Among the cellulose esters, there are the inorganic cellulose esters (cellulose nitrates, sulphates or phosphate . . . ), the organic cellulose esters (cellulose monoacetates, triacetates, amidopropionates, acetatebutyrates, acetatepropionates or acetatetrimellitates, etc.) and mixed organic/inorganic cellulose esters such as cellulose acetatebutyratesulphates and cellulose acetatepropionatesulphates. Among the cellulose ether esters, mention may be made of hydroxypropylmethylcellulose phthalates and ethylcellulose sulphates.

Among the non-ionic cellulose ethers, mention may be made of (C₁-C₄)alkylcelluloses such as methylcelluloses and ethylcelluloses (for example Ethocel standard 100 Premium from DOW CHEMICAL); (poly)hydroxy(C₁-C₄)alkylcelluloses such as hydroxymethylcelluloses, hydroxyethylcelluloses (for example Natrosol 250 HHR proposed by AQUALON) and hydroxypropylcelluloses (for example Klucel EF from AQUALON); mixed (poly)hydroxy(C₁-C₄)alkyl-(C₁-C₄)alkylcelluloses such as hydroxypropyl-methylcelluloses (for example Methocel E4M from DOW CHEMICAL), hydroxyethyl-methylcelluloses, hydroxyethyl-ethylcelluloses (for example Bermocoll E 481 FQ from AKZO NOBEL) and hydroxybutyl-methylcelluloses.

Among the anionic cellulose ethers, mention may be made of (poly)carboxy(C₁-C₄)alkylcelluloses and the salts thereof. By way of example, mention may be made of carboxymethylcelluloses, carboxymethylmethylcelluloses (for example Blanose 7M from the company AQUALON) and carboxymethylhydroxyethylcelluloses and the sodium salts thereof.

Among the cationic cellulose ethers, mention may be made of the cationic cellulose derivatives such as cellulose copolymers or cellulose derivatives grafted with a water-soluble quaternary ammonium monomer, and described in particular in the patent U.S. Pat. No. 4,131,576, such as (Poly)hydroxy(C₁-C₄)alkyl celluloses, such as hydroxymethyl-, hydroxyethyl- or hydroxypropyl celluloses grafted in particular with a methacryloylethyl-trimethylammonium, methacrylmidopropyl-trimethylammonium or dimethyl-diallylammonium salt. The marketed products corresponding to this definition are more particularly the products sold under the name “Celquat® L 200” and “Celquat® H 100” by the National Starch Company.

According to a preferred embodiment of the invention, the cellulosic polymer(s) are selected from cellulosic polymers not including a C₁₀-C₃₀ side fatty chain in the structure thereof; more preferably from cellulose ethers; more preferably still from non-ionic cellulose ethers; even most preferably from (a) (C₁-C₄)alkylcelluloses such as methylcelluloses and ethylcelluloses, (b) (poly)hydroxy(C₁-C₄)alkylcelluloses such as hydroxymethylcelluloses, hydroxyethylcelluloses and hydroxypropylcelluloses, (c) mixed (poly)hydroxy(C₁-C₄)alkyl-(C₁-C₄)alkylcelluloses such as hydroxypropyl-methylcelluloses, hydroxypropyl-ethylcelluloses, hydroxyethyl-methylcelluloses, hydroxyethyl-ethylcelluloses and hydroxybutyl-methylcelluloses, and (d) mixtures thereof.

More preferably, the composition according to the invention comprises at least one (poly)hydroxy(C₁-C₄)alkylcellulose such as hydroxymethylcelluloses, hydroxyethylcelluloses and hydroxypropylcelluloses; even most preferably at least hydroxyethylcellulose.

Preferably, when the composition according to the invention comprises at least one thickening agent, the total content of thickening agent(s) is comprised between 0.1 and 10% by weight, more preferably between 0.5 and 5% by weight, more preferably still between 1 and 2.5% by weight, relative to the total weight of the composition.

Preferably, when the composition according to the invention comprises at least one cellulosic polymer, the total content of cellulosic polymer(s) is comprised between 0.1 and 10% by weight, more preferably between 0.5 and 5% by weight, more preferably still between 1 and 2.5% by weight, relative to the total weight of the composition.

Preferably, when the composition according to the invention comprises at least one cellulosic polymer, the total content of (poly)hydroxy(C₁-C₄)alkylcellulose(s) is comprised between 0.1 and 10% by weight, more preferably between 0 5 and 5% by weight, more preferably still between 1 and 2.5% by weight, relative to the total weight of the composition.

Preferably, when the composition according to the invention comprises at least one cellulosic polymer, the total content of hydroxyethylcellulose is comprised between 0.1 and 10% by weight, more preferably between 0.5 and 5% by weight, more preferably still between 1 and 2.5% by weight, relative to the total weight of the composition.

Surfactants

The composition according to the invention may optionally further comprise at least one surfactant.

The surfactants that can be used according to the invention can be selected from the anionic surfactants, the cationic surfactants, the amphoteric and/or zwitterionic surfactants, the non-ionic surfactants, and mixtures thereof.

More preferably, the surfactant(s) which can be used according to the invention are selected from the non-ionic surfactants.

The non-ionic surfactants that can be used can be selected from alkylpolyglucosides (APG), oxyalkylenated glycerol esters, optionally oxyalkylenated fatty acid and sorbitan esters, polyoxyalkylenated (in particular polyoxyethylenated and/or polyoxypropylenated) fatty acid esters optionally in combination with a fatty acid and glycerol ester such as the PEG-100 Stearate/Glyceryl Stearate mixture marketed for example by the company ICI under the name Arlacel 165, oxyalkylenated sugar esters, and mixtures thereof.

As alkylpolyglucosides, mention may be made of those containing an alkyl group including 6 to 30 carbon atoms and preferably 8 to 16 carbon atoms, and containing a glucoside group preferably comprising 1.2 to 3 glucoside units. The alkylpolyglucosides can be selected, for example, from decylglucoside (Alkyl-C₉/C₁₁-polyglucoside (1.4)) such as the product marketed under the name Mydol 10® by the company Kao Chemicals or the product marketed under the name Plantacare 2000 UP® by the company Cognis; caprylyl/capryl glucoside, such as the product marketed under the name Plantacare KE 3711® by the company Cognis; laurylglucoside, such as the product marketed under the name Plantacare 1200 UP® by the company Cognis; cocoglucoside, such as the product marketed under the name Plantacare 818 UP® by the company Cognis; caprylylglucoside, such as the product marketed under the name Plantacare 810 UP® by the company Cognis; and mixtures thereof.

Oxyalkylenated glycerol esters are in particular the polyoxyethylenated derivatives of glyceryl and fatty acid esters and the hydrogenated derivatives thereof. These oxyalkylenated glycerol esters can be selected, for example, from hydrogenated and oxyethylenated glyceryl and fatty acid esters such as PEG-200 hydrogenated glyceryl palmate marketed under the name Rewoderm LI-S 80 by the company Goldschmidt; oxyethylenated glyceryl cocoates such as the PEG-7 glyceryl cocoate marketed under the name Tegosoft GC by the company Goldschmidt, and the PEG-30 glyceryl cocoate marketed under the name Rewoderm LI-63 by the company Goldschmidt; oxyethylenated glyceryl stearates; and mixtures thereof.

The oxyalkylenated sugar esters are in particular the polyethylene glycol ethers of fatty acid and sugar esters. These oxyalkylenated sugar esters can be selected, for example, from oxyethylenated glucose esters such as the PEG-120 methyl glucose dioleate marketed under the name Glucamate DOE 120 by the company Amerchol.

Preferably, the number of alkylene oxide moles of the non-ionic surfactants which can be used according to the invention ranges from 2 to 400; more preferably from 4 to 250.

Preferably, the composition according to the invention is free of surfactants.

According to a variant of the invention, the composition comprises at least one non-ionic surfactant; more preferably one non-ionic surfactant selected from the polyoxyethylenated glycerol esters; more preferably still at least one non-ionic surfactant selected from the hydrogenated and polyoxyethylenated glyceryl and fatty acid esters such as the PEG-200 hydrogenated glyceryl palmate, the polyoxyethylenated glyceryl cocoates such as the PEG-7 glyceryl cocoate and the PEG-30 glyceryl cocoate, the polyoxyethylated glyceryl stearates, and mixtures thereof.

More preferably still, according to this variant, the composition comprises at least one polyoxyethylenated glyceryl cocoate.

Preferably, when the composition according to the invention comprises at least one surfactant, the total content of surfactant(s) is comprised between 0.1 and 10% by weight, more preferably between 0.5 and 5% by weight, more preferably still between 1 and 4% by weight, relative to the total weight of the composition according to the invention.

Preferably, when the composition according to the invention comprises at least one surfactant, the total content of non-ionic surfactants (s) is comprised between 0.1 and 10% by weight, more preferably between 0.5 and 5% by weight, more preferably still between 1 and 4% by weight, relative to the total weight of the composition according to the invention.

Preferably, when the composition according to the invention comprises at least one surfactant, the total content of (poly)oxyethylenated glycerol esters is comprised between 0.1 and 10% by weight, more preferably between 0.5 and 5% by weight, more preferably between 1 and 4% by weight, relative to the total weight of the composition according to the invention.

Anti-Oxidant Agent

Preferably, the composition according to the invention is free of an anti-oxidant agent.

According to a variant of the invention, the composition further comprises at least one anti-oxidant agent; more preferably selected from tocopherol and the esters thereof, such as tocopherol acetate, propyl gallate, butylated hydroxytoluene (BHT), butylated hydroxyanisole (BHA), and mixtures thereof.

Sequestering Agent

Preferably, the composition according to the invention is free of a sequestering agent.

According to a variant of the invention, the composition further comprises at least one sequestering agent; more preferably selected from (a) ethylenediamine tetraacetic acid (EDTA) and the salts thereof such as the ethylenediamine tetraacetic acid disodium salt (Disodium EDTA), (b) the phosphonic derivatives and the salts thereof such as hexamethylene diamine tetra(methylene phosphonic acid), ethylenediamine tetra(methylene phosphonic acid), 1-hydroxyethylidene-1,1-diphosphonic acid, aminotri(methylenephosphonic acid), diethylene triamine penta(methylene phosphonic acid), (c) polyamine polymers such as polyalkylene polyamines and the derivatives thereof, in particular polyethyleneimine, (d) dendrimers with chelating activity, (e) proteins like spermine, spermidine, transferin, ferritin, (f) carboxylic acids such as phytic acid, citric acid, malic acid, nitriloacetic acid, fumaric acid, tartaric acid, succinic acid, oxalic acid, (g) the desferrioxamine mesylate, and mixtures thereof.

The definition of “sequestering agent” (also called “chelating agent”) is well known to the person skilled in the art and refers to a compound or a mixture of compounds capable of forming a chelate with a metal ion. A chelate is an inorganic complex in which a compound (the sequestering or chelating agent) is coordinated with a metal ion, that is to say that it forms one or more bonds with the metal ion (formation of a cycle including the metal ion).

A sequestering (or chelating) agent generally comprises at least two electron donor atoms which allow the formation of bonds with the metal ion.

According to another variant of the invention, the composition comprises at least one sequestering agent and at least one anti-oxidant agent.

According to another variant of the invention, the composition is free of fatty substance, sequestering agent and/or of anti-oxidant agent.

pH

Preferably, the pH of the composition, according to the invention, is comprised between 3 and 8, more preferably between 4 and 7, and even most preferably between 5 and 6.

The pH of these compositions can be adjusted to the desired value by means of alkalinizing agents or acidifying agents which are usually used. Among the basifying agents, mention may be made, by way of examples, of ammonia, alkanolamines, mineral or organic hydroxides. Among the acidifying agents, mention may be made, by way of examples, of mineral or organic acids such as hydrochloric acid, orthophosphoric acid, carboxylic acids such as, for example, acetic acid, tartaric acid, citric acid, lactic acid, sulphonic acids.

The use of amitriptyline and/or one of the pharmaceutically acceptable salts thereof as a preservative can be carried out in pharmaceutical compositions of different formulations, such as an aqueous gel, an aqueous solution, an oil-in-water emulsion, a cream, a milk, a lotion.

These compositions can be applied topically, as a drinkable solution, or else as injection, etc. Preferably, the composition is applied topically, and more preferentially onto the skin.

The present invention further relates to a pharmaceutical composition as defined above comprising amitriptyline and/or one of the pharmaceutically acceptable salts thereof, preferably in a total content comprised between 1 and 25% by weight, more preferably between 5 and 20% by weight, more preferably still between 5 and 15% by weight, most preferably between 10 and 15% by weight, relative to the total weight of the pharmaceutical composition, it being understood that the composition is as previously defined.

The composition may optionally further comprise at least one additional preservative, different from amitriptyline and the salts thereof, such as parabens (e.g., methyl paraben). In this case, the content of additional preservative(s) is preferably less than or equal to 10% by weight, more preferably less than or equal to 5% by weight, more preferably still less than or equal to 1% by weight, most preferably less than or equal to 0.5% by weight, relative to the total weight of the pharmaceutical composition.

In one particular embodiment, the composition according to the invention is free of additional preservative. In other words, amitriptyline and/or one of the pharmaceutically acceptable salts thereof is(are) the only preservative(s) in the composition according to the invention. In particular, preferably, the composition according to the invention is free of paraben, such as methyl paraben.

The following examples illustrate the composition according to the invention and the advantages of this composition. However, they in no way represent a limitation of the present invention but simply illustrate the invention.

### EXAMPLES

The compositions A (comparative) and B, C and D (according to the invention) were prepared from the ingredients indicated in the table below, whose amounts are expressed in % by weight:

TABLE 1

| | A | B | C | D |
|---|---|---|---|---|
| Amitriptyline | 0 | 5 | 10 | 10 |
| hydrochloride | | | | |
| Hydroxyethylcellulose | 1 | 1 | 1 | 1 |
| Propylene glycol | 5 | 5 | 5 | 5 |
| Methyl paraben | 0.1 | 0.1 | 0.1 | 0 |
| pH agent | Qs pH | Qs pH | Qs pH | Qs pH |
| | 5.5 ± 0.5 | 5.5 ± 0.5 | 5.5 ± 0.5 | 5.5 ± 0.5 |
| Water | Qs 100 | Qs 100 | Qs 100 | Qs 100 |

The compositions A, C and D were then subjected to antimicrobial preservation tests in accordance with European (Chapter 5.1.3 “Effectiveness of antimicrobial preservation”) and American (USP, Chapter 51) regulations. The studied microbial strains are indicated in the following table:

TABLE 2

| | European | |
|---|---|---|
| | Pharmacopoeia | USP |
| | Chapter 5.1.3 | Chapter 51 |
| Pseudomonas aeruginosa | X | X |
| Staphylococcus aureus | X | X |
| Escherichia coli | | X |
| Candida albicans | X | X |
| Aspergillus brasiliensis | X | X |

The enumeration of the microorganism growth (or the lack thereof) is performed at specified time intervals at 2, 7, 14 and 28 days and expressed as a logarithmic reduction relative to the control. The acceptance criteria according to European and American standards are presented in the following tables:

TABLE 3

| European Pharmacopoeia | | | | | | |
|---|---|---|---|---|---|---|
| criteria Chapter 5.1.3 | | | | | | |
| | logarithmic | | | | | |
| | concentration | | 2 | 7 | 14 | 28 |
| | in the | T | days | days | days | days |
| Microorganisms | inoculum | Criteria | Log reduction (CFU) | | | |
| P. aeruginosa | [5-6] | A | ≥2 | ≥3 | — | NI |
| S. aureus | [5-6] | B | — | — | ≥3 | NI |
| C. albicans | [5-6] | A | — | — | ≥2 | NI |
| A. brasiliensis | [5-6] | B | — | — | ≥1 | NI |

TABLE 4

| USP criteria Chapter 51 | | | | | | |
|---|---|---|---|---|---|---|
| | logarithmic | | | | | |
| | concentration | | 2 | 7 | 14 | 28 |
| | in the | T | days | days | days | days |
| Microorganisms | inoculum | Criteria | Log reduction (CFU) | | | |
| P. aeruginosa | [5-6] | USP | — | — | ≥2 | NI |
| S. aureus | | Criteria | | | | |
| E. coli | | | | | | |
| C. albicans | [5-6] | | — | — | NI | NI |
| A. brasiliensis | | | | | | |

T2, 7, 14, 28 days: results expressed as a logarithmic reduction compared to the control T
NI: No increase in the number of colonies relative to the previous count
T: logarithmic concentration of microorganisms in the inoculum used to carry out the contamination

10 g of each of the compositions A, C and D is inoculated with 100 μL of an inoculum of one of the tested microorganisms comprising 10⁷ to 10⁸ CFU/ml of inoculum for the bacteria and 10⁶ to 10⁷ CFU/ml of inoculum for the fungi.

Then, 1 g of composition inoculated by a microorganism is diluted in 9 mL of buffer solution before undergoing a series of dilutions and being distributed in Petri dishes.

Incubation for up to 28 days, at a temperature comprised between 30 and 35° C. for bacteria and between 20 and 25° C. for fungi.

The number of colonies is counted at 2, 7, 14 and 28 days.

The results are indicated in Tables 4 and 5 below:

TABLE 5

| | Microorganisms | | | | | | | |
|---|---|---|---|---|---|---|---|---|
| | S. aureus | | | | P. aeruginosa | | | |
| | Time (d) | | | | | | | |
| Composition | 2 | 7 | 14 | 28 | 2 | 7 | 14 | 28 |
| A | >4.7 | >4.7 | >4.7 | >4.7 | >4.7 | >4.7 | >4.7 | >4.7 |
| C | >4.7 | >4.7 | >4.7 | >4.7 | >4.7 | >4.7 | >4.7 | >4.7 |
| D | >4.7 | >4.7 | >4.7 | >4.7 | >4.7 | >4.7 | >4.7 | >4.7 |

TABLE 6

| | Microorganisms | | | | | | |
|---|---|---|---|---|---|---|---|
| | E. coli | | C. albicans | | A. brasiliensis | | |
| | Time (d) | | | | | | |
| Composition | 14 | 28 | 14 | 28 | 14 | 28 | Acceptance at 28 d |
| A | >4.7 | >4.7 | 1.7 | 2.9 | −0.4 | −0.4 | Non-compliant |
| | | | | | | | with USP, EP |
| C | >4.7 | >4.7 | >4.7 | >4.7 | 2 | 2 | Compliant with USP |
| | | | | | | | and EP criterion A |
| D | >4.7 | >4.7 | >4.7 | >4.7 | 1 | 1 | Compliant with USP |
| | | | | | | | and EP criterion B |

The composition A, comprising methyl paraben, but no amitriptyline, does not comply with European and American Pharmacopoeia tests.

The composition C, comprising methyl paraben and amitriptyline, complies with European and American Pharmacopoeia tests. The same applies to the composition D, comprising amitriptyline, but no methyl paraben.

Thus, the presence of amitriptyline allows reducing the populations of microorganisms such as P. aeruginosa, S. aureus, E. coli, C. albicans and A. brasiliensis, in pharmaceutical compositions, even in the absence of a preservative such as methyl paraben.

1. A method for the preservation of a pharmaceutical composition, comprising the addition of amitriptyline and/or one of the pharmaceutically acceptable salts thereof, into said pharmaceutical composition.

2. The method according to claim 1, characterised in that the total content of amitriptyline and/or one of the pharmaceutically acceptable salts is comprised between 1 and 25% by weight, relative to the total weight of the pharmaceutical composition.

3. The method according to claim 1, characterised in that the water content of the pharmaceutical composition is greater than or equal to 75% by weight, relative to the total weight of the pharmaceutical composition.

4. The method according to claim 1, characterised in that the pH of the pharmaceutical composition is between 3 and 8.

5. The method according to claim 1, characterised in that the composition further comprises at least one C₂-C₈ polyol.

6. The method according to claim 1, characterised in that the composition further comprises at least one thickening agent.

7. The method according to claim 1, characterised in that the composition further comprises at least one surfactant selected from anionic surfactants, cationic surfactants, amphoteric and/or zwitterionic surfactants, non-ionic surfactants, and mixtures thereof.

8. The method according to claim 1, characterised in that said composition is free of additional preservative.

9. The method according to claim 1, characterised in that the pharmaceutical composition is intended to be applied topically.
